(19) United States
(12) Patent Application Publication
(10) Pub. No.: US 2026/0356791 A1
(43) Pub. Date: Nov. 12, 2026

(54) CONCURRENT PRODUCTION OF HIGH PURITY MTBE AND HIGH PURITY ISOBUTYLENE WITH DIVIDING WALL COLUMN

(71) Applicant: Lummus Technology LLC, Houston, TX (US)

(72) Inventors: Rosette Barias, Houston, TX (US); Michael Jon Scott, Houston, TX (US)

(73) Assignee: Lummus Technology LLC, Houston, TX (US)

(21) Appl. No.: 19/246,464

(22) Filed: Jun. 23, 2025

Related U.S. Application Data
(60) Provisional application No. 63/662,979, filed on Jun. 21, 2024.

(51) Int. Cl. C07C 67/54; B01D 3/14; C07C 1/20; C07C 7/08

(52) U.S. Cl. CPC C07C 67/54; B01D 3/141; B01D 3/143; C07C 1/20; C07C 7/08

(57) ABSTRACT

A process for the coproduction of high purity methyl tertiary butyl ether and high purity isobutylene. The process includes feeding a crude MTBE stream into a dividing wall column. The crude MTBE stream is separated into a light hydrocarbon, a first side-stream, a second side-stream, and a heavy hydrocarbon. The first side-stream and a first portion of the second-side stream are fed to a reactor and decomposed, producing a raw IB stream. A second portion of the second side-stream is recovering as a high purity MTBE product. The raw IB stream and an extractant are fed to a methanol extraction column where an overhead washed reactor effluent and a bottoms product are recovered. The washed reactor effluent is fed to an IB dividing wall column and one or more of a light ends overhead, an high purity IB stream, and an IB bottom product.

### FIELD OF THE DISCLOSURE

Embodiments disclosed herein relate generally to production of methyl tertiary butyl ether (MTBE) and isobutylene (IB).

### BACKGROUND

Isobutylene is used as a raw material for the production of butyl rubber, PIB (polyisobutylene), MMA (methyl methacrylate) and is usually produced from the commercially proven MTBE decomposition process. MTBE is used in pharmaceutical applications. Embodiments disclosed herein are direct toward the concurrent production of high purity MTBE and high purity IB from all types of MTBE feedstocks using an MTBE decomposition process.

One process for producing high purity isobutylene from mixed C4 fractions is via back-cracking of methyl tertiary butyl ether (MTBE), such as described in U.S. Pat. No. 7,968,758. The isobutylene in the mixed butenes stream may be reacted with an alcohol, such as methanol, to form MTBE. After separation from the normal butenes, the MTBE may be decomposed to form isobutylene and methanol, allowing recovery of a high purity isobutylene stream. This process, however, involves many unit operations and frequent catalyst replacement.

U.S. Pat. No. 6,242,661 describes a process for the separation of isobutylene, otherwise inseparable from butene-1 by fractionation, in high purity. As described therein, the isobutylene is separated from butenes contained in a mixed hydrocarbon stream containing butene-1, butene-2 and small amounts of butadiene. The mixed hydrocarbon stream is fed to a distillation column reactor containing an alumina supported palladium oxide catalyst. The column is operated to tend to exclude butene-2 from contact with the catalyst and to maintain butene-1 in contact with the catalyst to isomerize the butene-1 to butene-2. As butene-2 is produced, it is distilled away from the catalyst, upsetting the equilibrium and allowing for a greater than equilibrium amount of butene-2. The isobutylene and isobutane are concurrently separated from the butene-2. Additionally, any butadiene in the feed may be hydrogenated to butenes. The bottoms is rich in butene-2, while the overheads, including isobutylene and isobutane, may be fed to a splitter for separation of the isobutylene from the isobutane.

### SUMMARY OF THE DISCLOSURE

In one aspect, embodiments disclosed herein relate a process for the coproduction of high purity methyl tertiary butyl ether (MTBE) and high purity isobutylene (IB). The process includes feeding a crude MTBE stream, comprising methanol, secondary butyl alcohol (SBA), tert-butyl alcohol (TBA), 2-methoxybutane (MSBE), diisobutylene (DIB), tertiary amyl methyl ether (TAME), and mixtures thereof, into a dividing wall column. The crude MTBE stream is separated into one or more of a light hydrocarbon, a first side-stream, a second side-stream, and a heavy hydrocarbon. The first side-stream and a first portion of the second-side stream are fed to to a reactor and decomposed, producing a raw IB stream comprising IB, methanol, and unreacted MTBE. A second portion of the second side-stream is recovering as a high purity MTBE product. The raw IB stream and an extractant are fed to a methanol extraction column where an overhead washed reactor effluent and a bottoms product are recovered. The washed reactor effluent is fed to an IB dividing wall column and one or more of a light ends overhead, an IB side-stream comprising high purity IB, and an IB bottom product comprising a mixture of IB, MTBE, MSBE, and DIB are recovered.

In another aspect, embodiments disclosed herein related to a process for the coproduction of high purity methyl tertiary butyl ether (MTBE) and high purity isobutylene (IB). The process includes feeding a crude MTBE stream, comprising methanol, secondary butyl alcohol (SBA), tert-butyl alcohol (TBA), 2-methoxybutane (MSBE), diisobutylene (DIB), tertiary amyl methyl ether (TAME), and mixtures thereof, into an MTBE topping column. The crude MTBE stream is separated into a light hydrocarbon comprising a mixture of C4s, C5s, and methanol, and an intermediate MTBE stream comprising a mixture of MTBE, TBA, MSBE, and higher olefins. The intermediate MTBE stream is separated in an MTBE tailing column and an MTBE stream and a heavy hydrocarbon stream are recovered. The MTBE stream is fed to a vapor compression system and a high purity MTBE stream and a reactor feed stream are received. The reactor feed stream is fed to a reactor and decomposed, producing a raw IB stream comprising IB, methanol, and unreacted MTBE. A second portion of the second side-stream is recovering as a high purity MTBE product. The raw IB stream and an extractant are fed to a methanol extraction column where an overhead washed reactor effluent and a bottoms product are recovered. The washed reactor effluent is fed to an IB dividing wall column and one or more of a light ends overhead, an IB side-stream comprising high purity IB, and an IB bottom product comprising a mixture of IB, MTBE, MSBE, and DIB are recovered.

The process flow diagram shown in the attached sketches can be slightly modified for specific crudes and product slates. Other aspects and advantages will be apparent from the following description and the appended claims.

### BRIEF DESCRIPTION OF DRAWINGS

FIG. 1 illustrates a simplified process flow diagram of systems and processes according to one or more embodiments disclosed herein.

FIG. 2 illustrates a simplified process flow diagram of systems and processes according to one or more embodiments disclosed herein.

FIG. 3 illustrates a simplified process flow diagram of systems and processes according to one or more embodiments disclosed herein.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before,” “after,” “single,” and other such terminology. Rather, the use of ordinal numbers is to distinguish between the elements. By way of an example, a first element is distinct from a second element, and the first element may encompass more than one element and succeed (or precede) the second element in an ordering of elements.

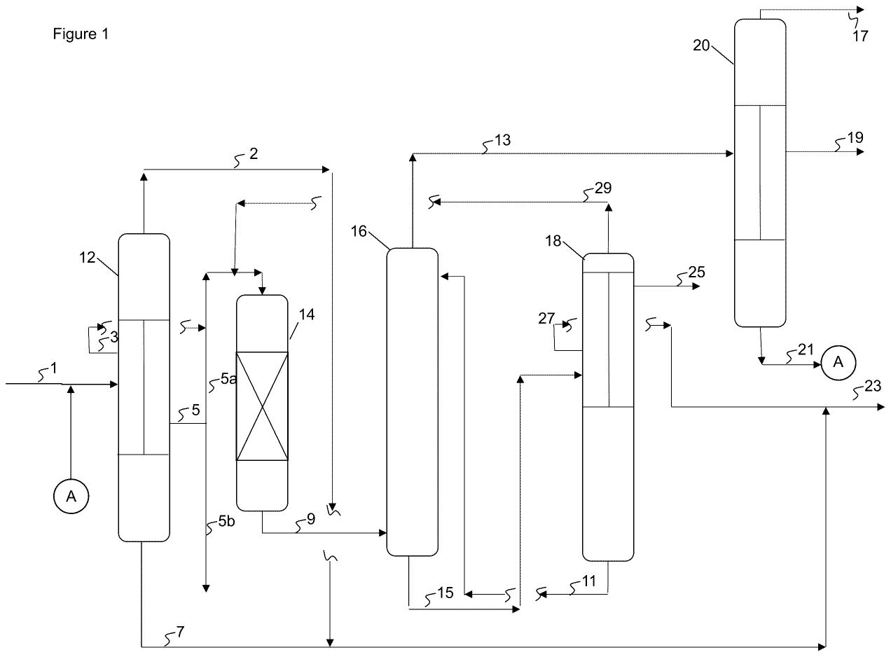

In the following description of FIGS. 1-3, any component described with regard to a figure, in various embodiments disclosed herein, may be equivalent to one or more like-named components described with regard to any other figure. For brevity, descriptions of these components will not be repeated with regard to each figure. Thus, each and every embodiment of the components of each figure is incorporated by reference and assumed to be optionally present within every other figure having one or more like-named components. Additionally, in accordance with various embodiments disclosed herein, any description of the components of a figure is to be interpreted as an optional embodiment which may be implemented in addition to, in conjunction with, or in place of the embodiments described with regard to a corresponding like-named component in any other figure.

Embodiments disclosed herein relate generally to a coproduction of high purity MTBE and high purity IB using a dividing wall column. Methods and systems for producing high purity IB from MTBE feed using multiple dividing wall columns are also provided. The method can include purifying the MTBE, decomposing the MTBE to produce IB and methanol, purifying the IB, and purifying methanol.

As used herein, the term “dividing wall column” refers to any column having a dividing wall suitable for the separation of a mixture containing two or more components having differing boiling points. As used herein, the term “dividing wall” refers to any partition disposed at least partially within an interior of a column to provide at least a first fractionation zone on one side of the dividing wall and a second fractionation zone on the other side of the dividing wall. The dividing wall can be either segmented or continuous. The dividing wall can be parallel or non-parallel relative to a longitudinal axis of the column. The first fractionation zone and the second fractionation zone can have the same or different cross-sectional areas and/or volumes. The column can have a circular cross-section and the dividing wall can positioned or disposed within the column to provide the first fractionation zone and the second fractionation zone having equal or un-equal cross-sectional areas with respect to one another. The dividing wall can extend completely or only partially from one side of the dividing wall column to the other side of the dividing wall column.

A dividing wall column is in principle a simplification of a system of thermally coupled distillation columns. In dividing wall columns, a dividing wall is located in the interior space of the column. A typical dividing wall column may use a chord wall or an annular wall. The dividing wall generally is vertical. Two different mass transfer separations occur on either side of the dividing wall, which may have different operating pressures and temperatures, the dividing wall may have to withstand a pressure differential and/or a temperature differential across the dividing wall.

Dividing wall columns may include internals which comprise trays, rotating trays, random and/or structured packings. Useful column trays include the following types: trays having drillholes or slots in the tray plate; trays having throats or chimneys which are covered by bubble-caps, caps or hoods; trays having drillholes in the tray plate which are covered by movable valves; trays having special constructions. In columns having rotating internals, the reflux is either sprayed by rotating funnels or distributed as a film onto a heated tube wall with the aid of a rotor. Columns may comprise random packings of various shaped bodies.

The method to produce high purity MTBE and high purity IB will now be described with reference to FIG. 1. To purify the MTBE, crude MTBE 1 is introduced into a dividing wall column 12. Crude MTBE 1 may be obtained from isobutenic C4 olefin mixtures, for example from the C4 cut from steam crackers or FCC units. The crude MTBE 1 may also include methanol, secondary butyl alcohol (SBA), tert-butyl alcohol (TBA), 2-methoxybutane (MSBE), diisobutylene, tertiary amyl methyl ether (TAME) and other high boiling point components.

In some embodiments, the crude MTBE 1 may have an MTBE content at a lower limit of 90 wt %, 91 wt %, 92 wt %, 93 wt %, 94 wt %, 95 wt % or 95.9 wt %, and an upper limit of 94 wt %, 95 wt %, 95.9 wt %, 96 wt %, 97 wt %, or 98 wt % MTBE, where any lower limit can be combined with any mathematically compatible upper limit. The crude MTBE 1 may also contain small amounts of highly unsaturated compounds such as 1,3-butadiene, trans-1,3-pentadiene, cis-1,3-pentadiene, 2-methyl-1,3-butadiene, and others.

The dividing wall column 12 can include a dividing wall disposed at least partially within the fractionation column such that an internal volume of the fractionation column is dividing into at least a pre-fractionation section and a main fractionation section. The pre-fractionation section and the main fractionation section can be in fluid communication with one another via a rectification section, a stripping section, or both. The rectification section can be defined between a top end of the fractionation column and a top end of the dividing wall. The stripping section can be defined between a bottom end of the fractionation column and a bottom end of the dividing wall.

The MTBE 1 can be introduced at an intermediate point in the pre-fractionation section of the dividing wall column 12. The intermediate point in the pre-fractionation section may be at a point along the length of the dividing wall column 12 and may be decided upon by one of ordinary skill in the art. A light hydrocarbon 2 can be withdrawn from the dividing wall column 12 at or proximal the top end thereof, a first side-stream 3 can be withdrawn from the pre-fractionation section, and a second side-stream 5 can be withdrawn from an intermediate point in the main fractionation section of the dividing wall column 12. A heavy hydrocarbon 7 can be withdrawn proximate the bottom end.

The intermediate point in the main fractionation section may be at a point along the length of the dividing wall column 12 and may be decided upon by one of ordinary skill in the art. The light hydrocarbon 2 may include C4s, C5s, and MeOH. The first intermediate fraction can include a composition similar to the crude MTBE 1, including MTBE and TBA. The second side-stream 5 may be a high purity MTBE stream. The heavy hydrocarbon 7 may be a mixture of MTBE, tert-butyl alcohol (TBA), 2-methoxybutane (MSBE) and higher olefins. The heavy hydrocarbon 7 may be collected as part of a liquid byproduct 23.

The dividing wall column 12 may operate at temperatures ranging from about 45 °C to about 130 °C and pressures ranging from about 0.1 to about 5 barg. The purification of MTBE 1 provides the second side stream 5 having a composition of about 99.5 wt % MTBE or greater, such as 99.8 wt % MTBE or 99.9 wt % MTBE,

The second side stream 5 having increased purity of MTBE, is produced by fractional distillation in the dividing wall column 12 by separating the MTBE 1 into the light hydrocarbons 2 comprising MTBE, methanol, and the heavy hydrocarbons 7 comprising butene oligomers and TBA.

To produce IB, the second side stream 5 may be split into a first portion 5a and a second portion 5b. The second portion 5b may be taken as high purity MTBE product. The first portion 5a may be sent to a reactor 14 to produce IB. The reactor 14 decomposes the pure MTBE in the second side stream 5 and produces a raw IB stream 9 comprising IB, methanol and unreacted MTBE, and others. In some embodiments, the reactor 14 includes a fixed bed.

The raw IB stream 9 is sent for product purification. The raw IB 9 may be sent to a methanol extraction column 16 to extract methanol from IB. The extraction column 16 uses an extractant 11 fed in a countercurrent fashion to the raw IB 9 thereby producing a washed reactor effluent 13 as an overhead and a bottoms product 15. The washed reactor effluent 13 may be fed to an IB dividing wall column 20 and the bottoms product 15 may be fed to a methanol dividing wall column 18. The extractant 11 may be water or another suitable extractant useful to separate methanol from IB.

To recover the IB, the washed reactor effluent 13 is fed to an IB dividing wall fractionation column 20, similar to that as described above for the purification of the MTBE. The washed reactor effluent 13 can be introduced at an intermediate point in the pre-fractionation section of the IB dividing wall fractionation column 20. The intermediate point in the pre-fractionation section may be at a point along the length of the IB dividing wall fractionation column 20 and may be decided upon by one of ordinary skill in the art. A light ends overhead 17 can be withdrawn from the IB dividing wall fractionation column 20 at or proximal the top end thereof and may be vented or recycled to the IB dividing wall fractionation column 20. The light ends overhead 17 may include one or more light components. A first side-stream of high purity IB 19 can be withdrawn from an intermediate point in the main fractionation section of the IB dividing wall fractionation column 20 as product. The intermediate point in the main fractionation section may be at a point along the length of the IB dividing wall column 20 and may be decided upon by one of ordinary skill in the art. The IB dividing wall column 20 also produces a bottom product 21 which may be a mixture of IB, MTBE, MSBE, and DIB which may recycled and mixed with MTBE 1 being fed to the dividing wall column 12.

To recover/purify the bottoms product 15 from the extraction column 16, the bottoms product 15 is fed to an intermediate point in the pre-fractionation section of the methanol dividing wall column 18. The intermediate point in the pre-fractionation section may be at a point along the length of the methanol dividing wall column 18 and may be decided upon by one of ordinary skill in the art. The methanol dividing wall column 18 provides a pure methanol product 25 withdrawn as a sidestream from an intermediate point in the main fractionation section of the methanol dividing wall column 18 and a bottoms stream 11. The bottoms stream 11 may include water and trace methanol. The intermediate point in the main fractionation section may be at a point along the length of the methanol dividing wall column 18 and may decided upon by one of ordinary skill in the art. The bottoms 11 may be recycled to the extraction column 16 as the extractant. A second sidestream 27 may also be collected from methanol dividing wall column 18. The second sidestream 27 may include TBA and be collected as part of the liquid by product 23 with the heavy hydrocarbon 7 from the MTBE dividing wall column 12.

The methanol dividing wall column 18 also produces an overhead product 29 which includes MTBE and MeOH. The overhead product 29 is recycled to the reactor 14 with the high purity MTBE 5.

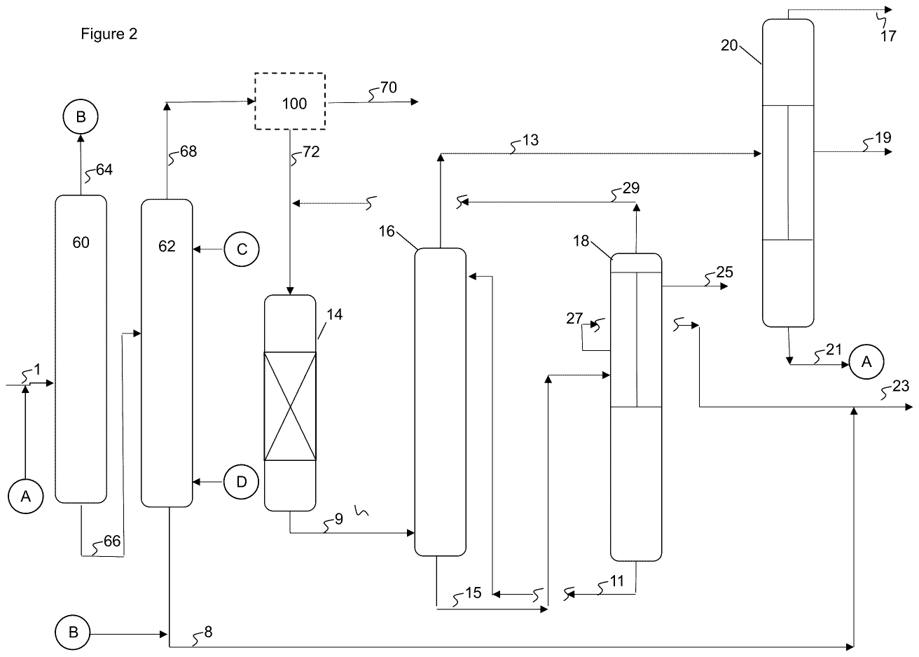

FIG. 2 illustrates another method to produce high purity MTBE and high purity IB. To purify the MTBE, crude MTBE 1 is introduced into an MTBE topping column 60. The MTBE 1 can be introduced at an intermediate point in the MTBE topping column 60. The intermediate point may be at a point along the length of the MTBE topping column 60 and may be decided upon by one of ordinary skill in the art. A light hydrocarbon 64 can be withdrawn from the MTBE topping column 60 at or proximal the top end thereof, an intermediate MTBE stream 66 can be withdrawn proximate the bottom end of the MTBE topping column 60.

The light hydrocarbon 64 may include C4s, C5s, and MeOH. The MTBE stream 66 may be a mixture of MTBE, tert-butyl alcohol (TBA), 2-methoxybutane (MSBE), and higher olefins.

The intermediate MTBE stream 66 is then fed to an MTBE heavies column 62. The MTBE heavies column may sometimes be referred to as an MTBE tailing column. An MTBE stream 68 may be recovered proximate the top of the MTBE heavies column 62 and a heavy hydrocarbon stream 8 may be recovered proximate the bottom of MTBE heavies column 62. The light hydrocarbon 64 and the heavy hydrocarbon 8 may be collected and combined with the second sidestream 27 as part of the liquid by product 23.

The MTBE stream 68 may then be fed to a vapor compression system 100. The vapor compression system 100 produces a pure MTBE stream 70 and an IB reactor 14 feedstock 72. Both the pure MTBE stream 70 and the IB reactor 14 feedstock 72 may have similar, or the same, composition.

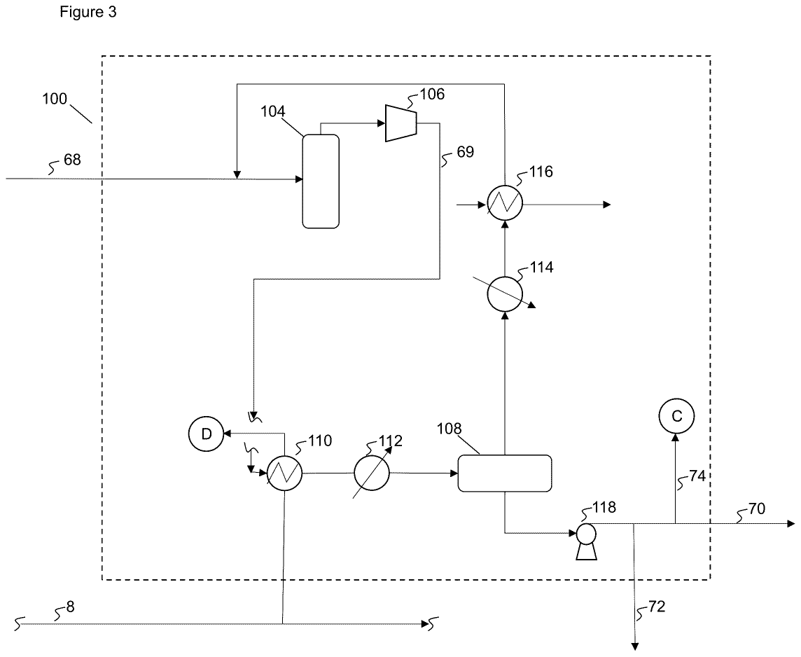

The vapor compression system 100, as illustrated in FIG. 3, includes a compressor knock out drum 104, compressor 106, overhead reflux drum 108, reboiler 110, trim cooler 112, trim heater 114, heat exchanger 116, and pump 118. The heat exchangers may be heat integrated with streams from downstream in the process, allowing for trim heater 114 to only be necessary at start-up. The vapor compression system 100 allows for a reduction in utilities cost while operating the MTBE heavies column 62. The overhead stream 68 is fed to the compressor knock out drum 104 and compressor 106, producing a compressed MTBE vapor 69. A portion of the column 62 bottoms stream 8 (FIG. 2) may be reboiled against the compressed vapor stream 69 and fed back to the MTBE heavies column 62 (FIG. 2). The compressed MTBE stream 69, after the reboiler 110, is fully cooled by trim cooler 112 and fed to the overhead reflux drum 108. Any remaining vapor fraction of the stream is recycled to the compressor knock out drum 104, while the remaining liquid portion is recovered as pure MTBE stream 70. A portion of the pure MTBE stream 70 may be recovered as the IB reactor 14 feedstock 72. Additionally, a portion 74 may be sent back to the MTBE heavies column 62 (FIG. 2) as reflux.

Returning now to FIG. 2, to produce IB, the IB reactor feedstock 72 is fed to the IB reactor 14 to produce IB. The remaining portion of the process is the same as with FIG. 1.

The overall processing schemes disclosed herein may be performed using dividing wall columns. Likewise, other resulting advantages may include: reduced capital investment; reduce operating costs, and elimination or significant reduction in the need for multiple columns to provide the same level of purity of products, among other advantages.

As described above, embodiments herein provide for the production of high purity isobutylene.

Unless defined otherwise, all technical and scientific terms used have the same meaning as commonly understood by one of ordinary skill in the art to which these systems, apparatuses, methods, processes and compositions belong.

The singular forms “a,” “an,” and “the” include plural referents, unless the context clearly dictates otherwise.

As used here and in the appended claims, the words “comprise,” “has,” and “include” and all grammatical variations thereof are each intended to have an open, non-limiting meaning that does not exclude additional elements or steps.

“Optionally” means that the subsequently described event or circumstances may or may not occur. The description includes instances where the event or circumstance occurs and instances where it does not occur.

When the word “approximately” or “about” are used, this term may mean that there can be a variance in value of up to ±10%, of up to 5%, of up to 2%, of up to 1%, of up to 0.5%, of up to 0.1%, or up to 0.01%.

Ranges may be expressed as from about one particular value to about another particular value, inclusive. When such a range is expressed, it is to be understood that another embodiment is from the one particular value to the other particular value, along with all particular values and combinations thereof within the range.

While the disclosure includes a limited number of embodiments, those skilled in the art, having benefit of this disclosure, will appreciate that other embodiments may be devised which do not depart from the scope of the present disclosure. Accordingly, the scope should be limited only by the attached claims.

What is claimed is:

1. A process for the coproduction of high purity methyl tertiary butyl ether (MTBE) and high purity isobutylene (IB) comprising:
feeding a crude MTBE stream, comprising methanol, secondary butyl alcohol (SBA), tert-butyl alcohol (TBA), 2-methoxybutane (MSBE), diisobutylene (DIB), tertiary amyl methyl ether (TAME), and mixtures thereof, into a dividing wall column;
separating the crude MTBE stream into one or more of a light hydrocarbon, a first side-stream, a second side-stream, and a heavy hydrocarbon;
feeding the first side-stream and a first portion of the second-side stream to a reactor and decomposing the first side-stream and the first portion of the second-side stream in the reactor, producing a raw IB stream comprising IB, methanol, and unreacted MTBE,
recovering a second portion of the second side-stream as a high purity MTBE product;
feeding the raw IB stream and an extractant to a methanol extraction column and recovering an overhead washed reactor effluent and a bottoms product;
feeding the washed reactor effluent to an IB dividing wall column and recover one or more of a light ends overhead, an IB side-stream comprising high purity IB, and an IB bottom product comprising a mixture of IB, MTBE, MSBE, and DIB.

2. The process of claim 1, wherein the crude MTBE has between 90 wt % and 96 wt % MTBE.

3. The process of claim 1, wherein the crude MTBE is introduced at an intermediate point in a pre-fractionation section of the dividing wall column.

4. The process of claim 1, wherein the light hydrocarbon comprising one or more of C4s, C5s, and MeOH, the first intermediate fraction comprises methanol, SBA, TBA, MSBE, DIB, and TAME, and the heavy hydrocarbon comprises one or more of MTBE, TBA, MSBE, and higher olefins.

5. The process of claim 1, further comprising recovering the heavy hydrocarbon as a first portion of a liquid byproduct.

6. The process of claim 1, further comprising operating the dividing wall column at temperatures ranging from 45 °C to 130 °C and pressures ranging from 0.1 to 5 barg.

7. The process of claim 1, further comprising feeding the bottoms product to a methanol dividing wall column and recovering one or more of the extractant, a methanol dividing wall column first side-stream comprising high purity methanol, a methanol dividing wall column second side-stream comprising TBA, and a methanol dividing wall column overheads stream comprising MTBE and methanol.

8. The process of claim 7, further comprising recycling the methanol dividing wall column overheads stream to the reactor with the first side-stream and the first portion of the second-side stream.

9. The process of claim 1, further comprising recycling the IB bottom product to the dividing wall column with the crude MTBE stream.

10. A process for the coproduction of high purity methyl tertiary butyl ether (MTBE) and high purity isobutylene (IB) comprising:
feeding a crude MTBE stream, comprising methanol, secondary butyl alcohol (SBA), tert-butyl alcohol (TBA), 2-methoxybutane (MSBE), diisobutylene (DIB), tertiary amyl methyl ether (TAME), and mixtures thereof, into an MTBE topping column;
separating the crude MTBE stream into a light hydrocarbon comprising a mixture of C4s, C5s, and methanol, and an intermediate MTBE stream comprising a mixture of MTBE, TBA, MSBE, and higher olefins;
separating the intermediate MTBE stream in an MTBE tailing column and recovering an MTBE stream and a heavy hydrocarbon stream;
feeding the MTBE stream to a vapor compression system and producing a high purity MTBE stream and a reactor feed stream;
feeding the reactor feed stream to a reactor and decomposing the reactor feed stream, producing a raw IB stream comprising IB, methanol, and unreacted MTBE,
feeding the raw IB stream and an extractant to a methanol extraction column and recovering an overhead washed reactor effluent and a bottoms product;
feeding the washed reactor effluent to an IB dividing wall column and recover one or more of a light ends overhead, an IB side-stream comprising high purity IB, and an IB bottom product comprising a mixture of IB, MTBE, MSBE, and DIB.

11. The process of claim 10, further comprising combining the heavy hydrocarbon stream and the light hydrocarbon as a liquid byproduct.

12. The process of claim 10, wherein the crude MTBE has between 90 wt % and 96 wt % MTBE.

13. The process of claim 10, further comprising feeding the bottoms product to a methanol dividing wall column and recovering one or more of the extractant, a methanol dividing wall column first side-stream comprising high purity methanol, a methanol dividing wall column second side-stream comprising TBA, and a methanol dividing wall column overheads stream comprising MTBE and methanol.

14. The process of claim 13, further comprising recycling the methanol dividing wall column overheads stream to the reactor with the reactor feed stream.

15. The process of claim 10, further comprising recycling the IB bottom product to the MTBE topping column with the crude MTBE stream.

16. The process of claim 10, wherein feeding the MTBE stream to a vapor compression system comprises:
feeding the MTBE stream to a compressor, producing a compressed MTBE vapor;
contacting, in indirect heat exchange, a portion of heavy hydrocarbon stream with the compressed vapor stream, producing a reboil stream and a cooled, compressed vapor stream;
feeding the reboil stream to the MTBE tailing column as a reboil liquid;
fully cooling the cooled, compressed vapor stream in a trim cooler, producing a fully cooled vapor stream;
feeding the fully cooled vapor stream to an overhead reflux drum;
recovering a first fraction from the overhead reflux drum as the reactor feed stream;
recovering a second fraction from the overhead reflux drum as the high purity MTBE stream; and
recovering a third fraction from the overhead reflux drum and recycling the third fraction to the compressor.
